(19) United States
(12) Patent Application Publication
(10) Pub. No.: US 2017/0360296 A1
(43) Pub. Date: Dec. 21, 2017

(54) OPTICAL MEASUREMENT APPARATUS FOR EYEBALL

(71) Applicant: FUJI XEROX CO., LTD., Tokyo (JP)

(72) Inventors: Junichiro HAYAKAWA, Kanagawa (JP); Kohei YUKAWA, Kanagawa (JP); Yoshinori SHIRAKAWA, Kanagawa (JP)

(73) Assignee: FUJI XEROX CO., LTD., Tokyo (JP)

(21) Appl. No.: 15/594,738

(22) Filed: May 15, 2017

(30) Foreign Application Priority Data
Jun. 15, 2016 (JP) 2016-119015

(51) Int. Cl. A61B 3/117; A61B 3/00; A61B 3/10; G02F 1/01

(52) U.S. Cl. CPC A61B 3/117; A61B 3/102; A61B 3/0075; A61B 3/0016; G02F 1/01

(57) ABSTRACT

An optical measurement apparatus for an eyeball, the optical measurement apparatus includes:
a light reflecting unit that reflects light in a direction where the light passes across an anterior chamber of the eyeball; and
a switching unit that switches an incident position of the light to the light reflecting unit so as to inhibit the light from being moved from a state in which the light passes across the anterior chamber.

### CROSS-REFERENCE TO RELATED APPLICATIONS

This application is based on and claims priority under 35 USC 119 from Japanese Patent Application No. 2016-119015 filed Jun. 15, 2016.

### BACKGROUND

### Technical Field

The present invention relates to an optical measurement apparatus for an eyeball.

### SUMMARY

According to an aspect of the invention, an optical measurement apparatus for an eyeball includes:

a light reflecting unit that reflects light in a direction where the light passes across an anterior chamber of the eyeball; and

a switching unit that switches an incident position of the light to the light reflecting unit so as to inhibit the light from being moved from a state in which the light passes across the anterior chamber.

### BRIEF DESCRIPTION OF THE DRAWINGS

Exemplary embodiments of the present invention will be described in detail based on the following figures, wherein:

FIGS. 1A and 1B are views illustrating an example of a configuration of an optical measurement apparatus for an eyeball to which a first exemplary embodiment is applied, in which FIG. 1A is a view obtained when the eyeball is viewed from the top side, and FIG. 1B is a view obtained when the eyeball is viewed from the front side;

FIGS. 2A and 2B are views illustrating an exemplary configuration of an optical measurement apparatus for an eyeball to which a second exemplary embodiment is applied, in which FIG. 2A is a view obtained when the eyeball is viewed from the top side, and FIG. 2B is a view when the eyeball is viewed from the front side;

FIGS. 3A and 3B are views illustrating an exemplary configuration of an optical measurement apparatus for an eyeball to which a third exemplary embodiment is applied, in which FIG. 3A is a view obtained when the eyeball is viewed from the top side, and FIG. 3B is a view of the eyeball obtained when the eyeball is viewed from the front side;

FIG. 4 is a view for explaining a method of measuring, by the optical measurement apparatus, a rotation angle (optical rotation) of a vibrating surface caused by an optically active substance included in an aqueous humor in an anterior chamber;

FIGS. 5A and 5B are views illustrating an exemplary configuration of an optical measurement apparatus for an eyeball to which a fourth exemplary embodiment is applied, in which FIG. 5A is a view obtained when the eyeball is viewed from the top side, and FIG. 5B is a view obtained when the eyeball is viewed from the front side;

FIGS. 6A and 6B are views illustrating an exemplary configuration of an optical measurement apparatus for an eyeball to which a fifth exemplary embodiment is applied, in which FIG. 6A is a view obtained when the eyeball is viewed from the top side, and FIG. 6B is a view obtained when the eyeball is viewed from the front side; and

FIGS. 7A and 7B are views illustrating an exemplary configuration of an optical measurement apparatus for an eyeball to which a sixth exemplary embodiment is applied, in which FIG. 7A is a view obtained when the eyeball is viewed from the top side, and FIG. 7B is a view obtained when the eyeball is viewed from the front side.

### DETAILED DESCRIPTION

Hereinafter, exemplary embodiments of the present invention will be described with reference to the accompanying drawings. Further, the accompanying drawings illustrate an eyeball such that the eyeball is greater than other members (such as an optical system to be described below) or smaller than the other members (such as the optical system to be described below) in order to clearly define a relationship between the eyeball and an optical path.

### (Background of Measuring Glucose Concentration of Aqueous Humor)

First, a background of measuring the glucose concentration of an aqueous humor will be described.

It is recommended that a type 1 diabetic patient and a type 2 diabetic patient (measurement subject), who require insulin therapy, perform a self-blood glucose monitoring. To perform the self-blood glucose monitoring, the measurement subject measures his/her blood glucose level by him/herself at home or the like in order to precisely control the blood glucose.

A self-blood glucose monitor, which is currently distributed, measures the glucose concentration in the blood by puncturing a fingertip or the like by using a needle and then collecting a minute amount of blood. It is frequently recommended that the patient performs the self-blood glucose monitoring after each meal or before going to bed, and it is required to perform the self-blood glucose monitoring once to several times a day. In particular, in the case of intensive insulin therapy, it is required to perform the measurement much more times.

For this reason, an invasive blood glucose level monitoring method using a puncture-type self-blood glucose monitor easily causes a deterioration in incentives in respect to the self-blood glucose monitoring of the measurement subject due to the pain caused by a test (puncture by a pin prick) when collecting the blood. For this reason, in some cases, it is difficult to efficiently treat diabetes.

Therefore, a non-invasive blood glucose level monitoring method is being developed which does not requires the puncture, instead of the invasive blood glucose monitoring method such as the puncture.

As a non-invasive blood glucose level monitoring method, a near infrared spectroscopy method, an optoacoustic spectroscopy method, a method of using optical rotation, and the like are discussed. Further, these methods estimate a blood glucose level based on a glucose concentration.

The near infrared spectroscopy and the optoacoustic spectroscopy detect light absorption spectrum or acoustic vibration of the blood in a blood vessel of a finger. However, cell substances such as erythrocytes and leukocytes are present in the blood. For this reason, the near infrared spectroscopy method and the optoacoustic spectroscopy method are greatly affected by light scattering. Further, the near infrared spectroscopy method and the optoacoustic spectroscopy method are also affected by tissue at a circumference of the blood vessel as well as the blood in the blood vessel. Therefore, in these methods, it is difficult to separate signals because it is necessary to detect a signal with respect to a glucose concentration based on signals associated with enormous substances such as protein, amino acid, and the like.

Meanwhile, the aqueous humor in the anterior chamber has almost the same substances as serum, and includes protein, glucose, ascorbic acid, and the like. However, the aqueous humor does not include cell substances such as erythrocyte and leukocytes unlike the blood, and thus the aqueous humor is less affected by light scattering. Therefore, the aqueous humor is suitable for the optical measurement of a glucose concentration.

Therefore, the concentration of optically active substances including glucose may be optically measured by using the aqueous humor.

In addition, protein, glucose, ascorbic acid, and the like included in the aqueous humor are optically active substances, and have optical rotation. Therefore, the concentration of optically active substances including glucose may be optically measured by using the optical rotation.

Because the aqueous humor is a tissue fluid for transporting the glucose, it is considered that the glucose concentration in the aqueous humor is associated with the glucose concentration in the blood. Further, it is reported that in the measurement using a rabbit, a length of time required to transport the glucose from the blood to the aqueous humor (transportation delay time) is within 10 minutes.

As described above, the glucose concentration in the blood is obtained by measuring the glucose concentration in the aqueous humor.

By the way, in a technique of optically measuring the concentration of an optically active substance such as glucose included in an aqueous humor, the following two optical paths may be set.

One is an optical path in which light is caused to enter the eyeball at an angle approximately perpendicular to the eyeball, that is, in a front-back direction, the light is caused to be reflected by an interface between a cornea and the aqueous humor or an interface between the aqueous humor and a crystalline lens, and the reflected light is received (detected). The other is an optical path in which light is caused to enter the eyeball at an angle approximately parallel to the eyeball, and the light, which is caused to pass through across the anterior chamber, is received (detected).

In the case of the optical path like the former optical path in which the light is caused to enter the eyeball at an angle perpendicular to the eyeball, there is a concern that the light may reach a retina. In particular, in a case in which a laser having a high coherency is used as a light source, there is a concern that the light may reach the retina.

On the contrary, in the case of the optical path like the latter optical path in which light is caused to enter the eyeball at an angle approximately parallel to the eyeball, and to pass through the anterior chamber while traversing the anterior chamber, the light is inhibited from reaching the retina.

The concentration of the optically active substance or the optical rotation depends on the length of the optical path, and the longer the optical path is, the higher the optical rotation is. Therefore, since the light passes through across the anterior chamber, the length of the optical path may be set to be long.

As described above, the optical path in which the light is caused to pass through across the anterior chamber is adopted here.

### First Exemplary Embodiment

### <Optical Measurement Apparatus 1>

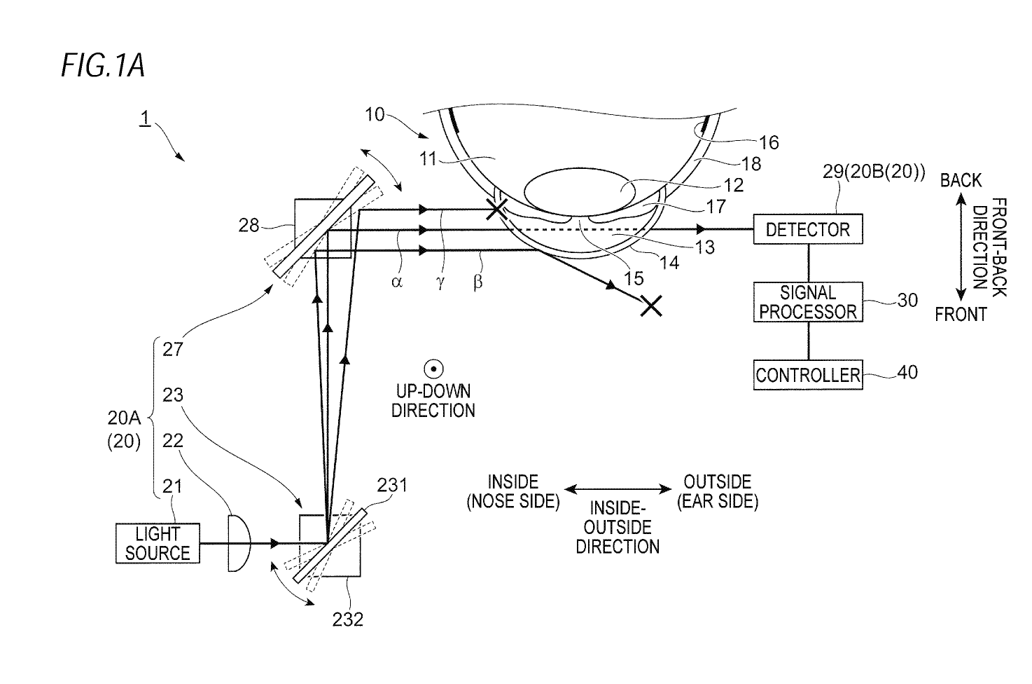

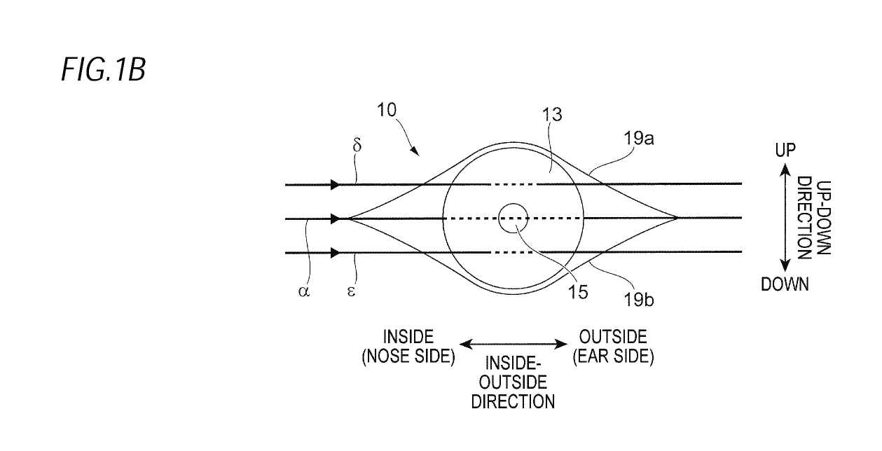

FIGS. 1A and 1B are views illustrating an exemplary configuration of an optical measurement apparatus 1 for an eyeball to which a first exemplary embodiment is applied. FIG. 1A is a view obtained when an eyeball 10 is viewed from the top side (a cross-sectional view in upward and downward directions), and FIG. 1B is a view obtained when the eyeball 10 is viewed from the front side. Further, it is assumed that the eyeball 10 illustrated in FIGS. 1A and 1B is a left eye. FIGS. 1A and 1B illustrate, by arrows, an inside-outside direction which indicates an inside of a face (nose side) and an outside of the face (ear side), an front-back direction which indicates front and back sides of the face, and an up-down direction which indicates the upper and lower sides of the face.

The optical measurement apparatus 1 for an eyeball (hereinafter, referred to as an “optical measurement apparatus 1”) includes an optical system 20 which is used to measure a property of an aqueous humor in an anterior chamber 13 (to be described below) of the eyeball (subject's eye) 10 of a measurement subject (test subject), a signal processor 30 which processes signals obtained by the optical system 20, and a controller 40 which controls the optical system 20.

The optical measurement apparatus 1 to which the first exemplary embodiment is applied measures the concentration of an optically active substance included in the aqueous humor based on the light intensity of transmitted light transmitted through the aqueous humor.

First, the structure of the eyeball 10 will be described.

As illustrated in FIG. 1A, the eyeball 10 has a substantially spherical external shape, and has a vitreous body 11 at the center thereof. Further, a rear half of the eyeball 10 is omitted from FIG. 1A. Further, a crystalline lens 12, which serves as a lens, is buried in a part of the vitreous body 11. The anterior chamber 13 is present at the front side of the crystalline lens 12, and a cornea 14 is present at the front side of the anterior chamber 13. The anterior chamber 13 and the cornea 14 protrude convexly from the spherical shape.

The peripheral portion of the crystalline lens 12 is surrounded by an iris 17, and a pupil 15 is present at the center of the iris 17. The vitreous body 11 is covered by a retina 16, except for a portion in contact with the crystalline lens 12. Further, the retina 16 is covered by a sclera 18. That is, an outside of the eyeball 10 is covered by the cornea 14 and the sclera 18.

The anterior chamber 13 is a region surrounded by the cornea 14 and the crystalline lens 12. The anterior chamber 13 has a circular shape when viewed from the front side (see FIG. 1B). Further, the anterior chamber 13 is filled with the aqueous humor.

As illustrated in FIG. 1B, the surface of the eyeball 10 is covered by an upper eyelid 19a and a lower eyelid 19b.

Next, the optical system 20 will be described.

As illustrated in FIG. 1A, the optical system 20 includes a light emitting system 20A which emits light toward the anterior chamber 13 of the eyeball 10, and a light receiving system 20B which receives light passing through the anterior chamber 13.

First, the light emitting system 20A includes a light source 21, a collimator lens 22, a deflector 23, and a mirror 27 as an example of a light reflecting unit.

The light source 21 may be a light source having a wide wavelength width like a light emitting diode (LED) or a lamp, or may be a light source having a narrow wavelength width like a laser. In addition, the light source 21 may have plural LEDs, lamps, or lasers. Further, the light source 21 may use plural wavelengths.

The collimator lens 22 converts a light beam, which is projected from the light source 21 and has an area, into parallel light beams each having a small diameter. Because the anterior chamber 13 is a small region surrounded by the cornea 14 and the crystalline lens 12, it is more desirable that the light beams to be transmitted through the anterior chamber 13 have small diameters.

At this time, when the light projected from the light source 21 has a small diameter, it is not necessary to use the collimator lens 22.

The deflector 23 refers to a member that deflects a direction in which the light travels, and for example, the deflector 23 includes a mirror 231, and a driving device 232 which changes the inclination of a reflecting surface of the mirror 231. The mirror 231 may be a galvano mirror or a polygon mirror. In the case of the galvano mirror, the inclination of a reflecting surface is changed by rotating the reflecting surface about an axis provided on the reflecting surface. In the case of the polygon mirror, the inclination of a reflecting surface is changed by rotating a polyhedral mirror. The galvano mirror or the polygon mirror deflects the light in a one-dimensional direction because the reflecting surface is inclined in one direction (one-dimensional direction).

The mirror 231 may be a mirror configured with micro electro mechanical systems (MEMS). In a case in which the reflecting surface is configured to be inclined with respect to a point, the reflecting surface is inclined in one direction and a direction orthogonal to the one direction. Therefore, because the reflecting surface is inclined in a two-dimensional direction, the light is deflected in the two-dimensional direction.

The inclination of the mirror 231 is controlled by the driving device 232. In a case in which the mirror 231 is the galvano mirror or the polygon mirror, the driving device 232 includes, for example, a motor, and a circuit that controls the motor. In addition, in a case in which the mirror 231 is configured with the MEMS, the driving device 232 is a driving circuit that is configured integrally with the mirror 27 and supplies an electric potential to plural electrodes that control the inclination of the mirror 27 by using an electrostatic force.

The mirror 231 is an example of a reflective member, and the driving device 232 is an example of an angle change unit.

The mirror 27 reflects the light deflected by the deflector 23 so that the light passes across the anterior chamber 13. In the first exemplary embodiment, similar to the deflector 23, the mirror 27 is connected to a driving device 28. The mirror 27 is a galvano mirror, a polygon mirror, or a mirror configured with the MEMS. Further, the inclination of the mirror 27 is changed by the driving device 28 so as to change the reflection angle with respect to the incident light.

Here, the deflector 23 and the driving device 28 are an example of a switching unit.

The light receiving system 20B includes a detector 29. Here, the detector 29 is a light receiving element such as, for example, a silicon diode. The detector 29 converts the intensity of the light passing through the anterior chamber 13 into an electrical signal.

The signal processor 30 receives the electrical signal from the detector 29 and processes the electrical signal so as to calculate the concentration of the optically active substance included in the aqueous humor.

As described above, the controller 40 controls the optical system 20 and the signal processor 30.

Next, a relationship between the eyeball 10 and the optical system 20 will be described.

First, as illustrated in FIG. 1A, the optical system 20 is set with respect to the eyeball 10 such that light projected from the light emitting system 20A is caused to enter the light receiving system 20B through an optical path indicated by an optical path α. That is, as illustrated in FIG. 1A, the optical path α passes through the central portion of the anterior chamber 13 when viewing the eyeball 10 in a cross-sectional view in the up-down direction. Further, as illustrated in FIG. 1B, the optical path α passes through the central portion of the anterior chamber 13 even when viewing the eyeball 10 from the front side.

The optical path α is an optical path suitable to measure the concentration of the optically active substance included in the aqueous humor in the anterior chamber 13.

In addition, an optical path β illustrated in FIG. 1A is an optical path which is too much forward of the eyeball 10 and is reflected by the surface of the cornea 14. The optical path β does not pass through the aqueous humor in the anterior chamber 13. In addition, an optical path γ is an optical path which is too much rearward of the eyeball 10 and is blocked by the iris 17 or the sclera 18. The optical path γ does not pass through the aqueous humor in the anterior chamber 13.

An optical path δ illustrated in FIG. 1B is an optical path which is too much upward of the eyeball 10, and a length by which the optical path δ passes through the aqueous humor in the anterior chamber 13 is short. Further, if an optical path is further too much upward of the eyeball 10 as compared with the optical path δ, the optical path is blocked by the upper eyelid 19a and does not pass through the aqueous humor in the anterior chamber 13.

An optical path ε is an optical path which is much downward of the eyeball 10, and a length by which the optical path ε passes through the aqueous humor in the anterior chamber 13 is short. Further, if an optical path is further too much downward of the eyeball 10 as compared to the optical path ε, the optical path is blocked by the lower eyelid 19b and does not pass through the aqueous humor in the anterior chamber 13.

The terms “optical paths α, β, γ, δ, and ε” are used to explain the states and positions of the optical paths with respect to the anterior chamber 13 of the eyeball 10.

However, in some cases, the optical paths may deviate because a relative position relationship between the eyeball 10 and the optical system 20 or a shape of the cornea 14 is changed over time, and as a result, a state of the optical path α may not be maintained. Further, the eyeball 10 may be moved relative to the optical system 20, and the optical system 20 may be moved relative to the eyeball 10. Hereinafter, for convenience, the description will be made assuming that the eyeball 10 is moved relative to the optical system 20.

In a case in which an optical path, which is in the state of the optical path α, is brought into the state of the optical path β or the optical path γ or brought into a state of the optical path δ or the optical path ε with respect to the eyeball 10, that is, in a case in which the optical path slightly deviates, the state of the optical path may return back to the state of the optical path α by displacing (moving) or switching the optical path. That is, it is not necessary to set the optical system 20 again with respect to the eyeball 10.

For example, as illustrated in FIG. 1A, it is assumed that an optical path, which is in the state of the optical path α, is brought into the state of the optical path β because the eyeball 10 moves rearward. In this case, a new optical path may be set at a position of the optical path γ. Therefore, based on a control by the controller 40, the deflector 23 sets an optical path to a position of the optical path γ by switching an incident position to the mirror 27. That is, an incident position of light is switched to the mirror 27 in order to set an optical path to the position of the optical path γ from the position of the optical path α, so that the position of the optical path γ is reset to the state of the optical path α which is suitable to measure the concentration of the optically active substance included in the aqueous humor in the anterior chamber 13.

Similarly, it is assumed that an optical path, which is in the state of the optical path α, is brought into the state of the optical path γ because the eyeball 10 moves forward. In this case, a new optical path may be set at the position of the optical path β. Therefore, based on a control by the controller 40, the deflector 23 sets an optical path to the position of the optical path β by switching an incident position to the mirror 27. That is, the incident position of light is switched to the mirror 27 in order to set an optical path to the position of the optical path β from the position of the optical path α, so that the position of the optical path β is reset to the state of the optical path α which is suitable to measure the concentration of the optically active substance included in the aqueous humor in the anterior chamber 13.

As illustrated in FIG. 1B, it is assumed that an optical path, which is in the state of the optical path α, is brought into the state of the optical path ε because the eyeball 10 moves upward. In this case, a new optical path may be set at the position of the optical path δ. Therefore, based on a control by the controller 40, the deflector 23 sets an optical path to the position of the optical path δ by switching an incident position to the mirror 27. That is, the incident position of light is switched to the mirror 27 in order to set an optical path to the position of the optical path δ from the position of the optical path α, so that the position of the optical path δ is reset to the state of the optical path α which is suitable to measure the concentration of the optically active substance included in the aqueous humor in the anterior chamber 13.

Similarly, it is assumed that an optical path, which is in the state of the optical path α, is brought into the state of the optical path δ because the eyeball 10 moves downward. In this case, a new optical path may be set at the position of the optical path ε. Therefore, based on a control by the controller 40, the deflector 23 sets an optical path to the position of the optical path ε by switching the incident position to the mirror 27. That is, the incident position of light is switched to the mirror 27 in order to set an optical path to the position of the optical path ε from the position of the optical path α, so that the position of the optical path ε is reset to the state of the optical path α which is suitable to measure the concentration of the optically active substance included in the aqueous humor in the anterior chamber 13.

In this case, the incident angle of light is changed by changing the inclination (incident angle of light) of the mirror 231 of the deflector 23 and the inclination (incident angle of light) of the mirror 27.

In FIGS. 1A and 1B, the optical paths run in parallel. This is because a relative positional relationship between the light emitting system 20A and the light receiving system 20B of the optical system 20 is maintained. The optical paths may not necessarily run in parallel.

The mirror 231 or the mirror 27 is described as being a plane mirror, but may be a concave mirror, a convex mirror, a spherical mirror, a parabolic mirror, and the like.

As described above, in the optical measurement apparatus 1 of the first exemplary embodiment, even though an optical path, which is in the state of the optical path α, deviates because a relative position relationship between the eyeball 10 and the optical system 20, a shape of the cornea 14, or the like is changed over time, the optical path is reset to the state of the optical path α, which is suitable to measure the concentration of the optically active substance included in the aqueous humor in the anterior chamber 13, by switching the incident position of light to the mirror 27. That is, the optical path is set to pass across the anterior chamber 13.

The state in which the optical path deviates from the state of the optical path α may be easily detected by the signal processor 30 that receives a signal from the detector 29. Therefore, based on the signal from the signal processor 30, the controller 40 may control the inclination (incident angle of light) of the mirror 231 of the deflector 23 and the inclination (incident angle of light) of the mirror 27.

The incident angle of the mirror 231 of the deflector 23 and the incident angle of the mirror 27 may be changed not only in the inside-outside direction and but also in the up-do direction. In a case in which the incident angle is changed in the inside-outside direction, the incident position of light is switched to the mirror 27 in a one-dimensional direction in the front-back direction (among the optical paths α, β, and γ). In addition, in a case in which the incident angle is changed in the up-down direction, the incident position of light is switched to the mirror 27 in a one-dimensional direction in the up-down direction (among the optical paths α, δ, and ε). In a case in which the incident angle is changed in the front-back direction and the up-down direction, the incident position of light is switched to the mirror 27 in a two-dimensional direction in the front-back direction (among the optical paths α, β, and γ) and the up-down direction (among the optical paths α, δ, and ε).

### Second Exemplary Embodiment

In the first exemplary embodiment, the incident angle of light is changed by the mirror 27 in addition to the mirror 231 of the deflector 23.

In the second exemplary embodiment, the incident angle of light to the mirror 27 is fixed.

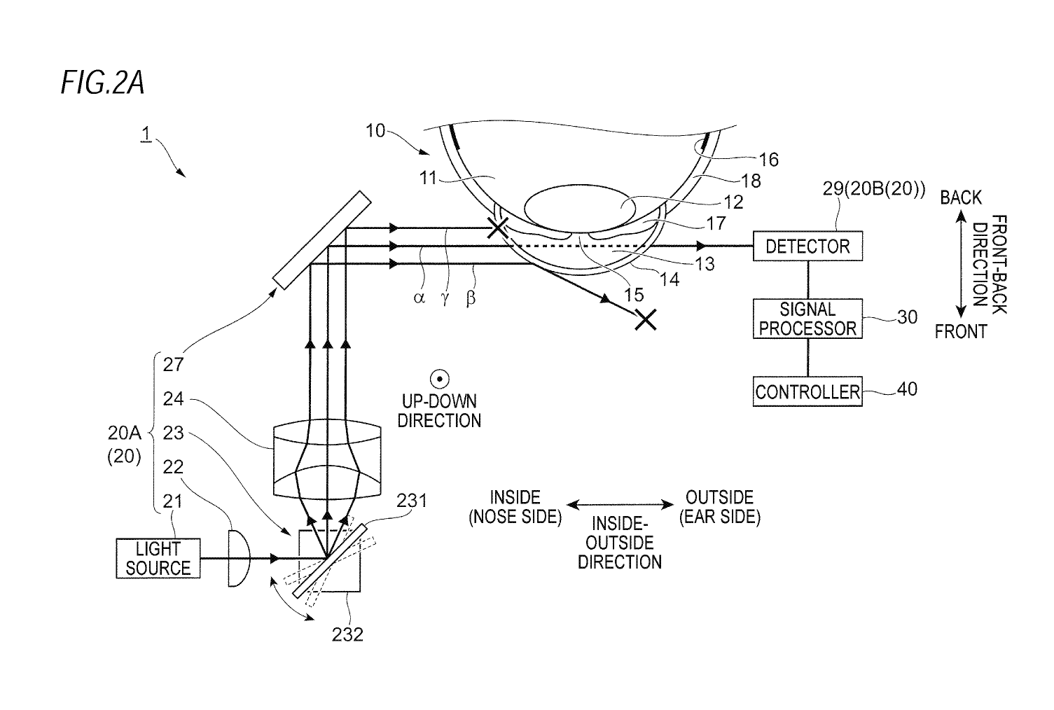

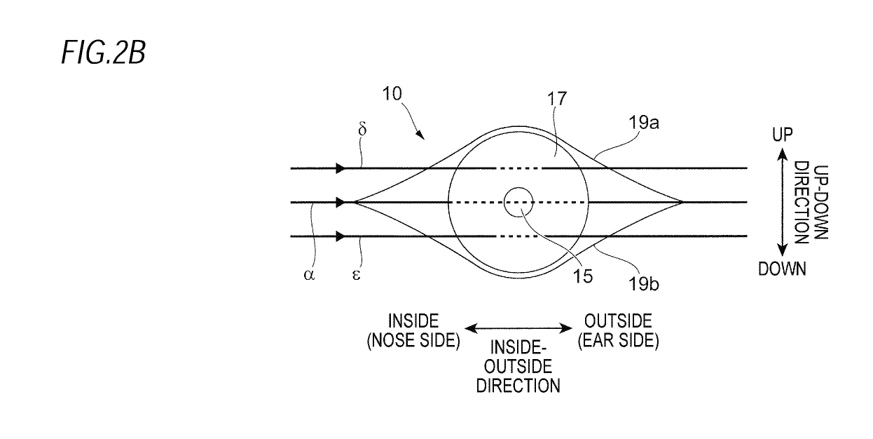

FIGS. 2A and 2B are views illustrating an exemplary configuration of the optical measurement apparatus 1 for an eyeball to which the second exemplary embodiment is applied. FIG. 2A is a view obtained when the eyeball 10 is viewed from the top side (a cross-sectional view in the upward and downward directions), and FIG. 2B is a view obtained when the eyeball 10 is viewed from the front side. The parts similar to those of the optical measurement apparatus 1 to which the first exemplary embodiment is applied are designated by the same reference numerals, and description thereof will be omitted.

In the optical measurement apparatus 1 for an eyeball to which the second exemplary embodiment is applied, a telecentric optical system 24 including a telecentric fθ lens is provided between the deflector 23 and the mirror 27. Further, the mirror 27 does not have the driving device 28 provided in the first exemplary embodiment. Here, the deflector 23 and the telecentric optical system 24 are an example of a switching unit.

The telecentric fθ lens is a lens that condenses an incident light beam in a direction perpendicular to a flat surface. That is, as illustrated in FIGS. 2A and 2B, even though light beams are reflected by the mirror 231 of the deflector 23 and then obliquely enter the telecentric optical system 24, the light beams are projected from the telecentric optical system 24 in parallel with each other.

Therefore, even though an incident angle (inclination) of the mirror 27 is fixed, the optical paths, which run toward the eyeball 10, are changed to be parallel to each other so that the optical paths run in parallel with each other by switching an incident position to the mirror 27.

Therefore, an incident position to the mirror 27 may be switched by controlling the reflection angle of the mirror 231 of the deflector 23. That is, the control of switching an incident position to the mirror 27 is simplified.

Because the mirror 27 is provided to be close to the eyeball 10, in the optical measurement apparatus 1 to which the first exemplary embodiment is applied, a dynamic force is applied to the measurement subject when the mirror 27 is moved (rotated) to change the incident angle of the mirror 27. However, because the incident angle of the mirror 27 is fixed in the optical measurement apparatus 1 to which the second exemplary embodiment is applied, a dynamic force is inhibited from being applied to the measurement subject.

Because the operation of switching an incident position to the mirror 27 is identical to that described in the first exemplary embodiment except that the incident angle (inclination) of the mirror 27 is fixed, a description thereof will be omitted.

### Third Exemplary Embodiment

In the first exemplary embodiment and the second exemplary embodiment, the concentration of the optically active substance included in the aqueous humor is measured based on a change in intensity of the light transmitted through the aqueous humor in the anterior chamber 13.

In the third exemplary embodiment, the concentration of the optically active substance such as glucose included in the aqueous humor is measured by using an optical rotation.

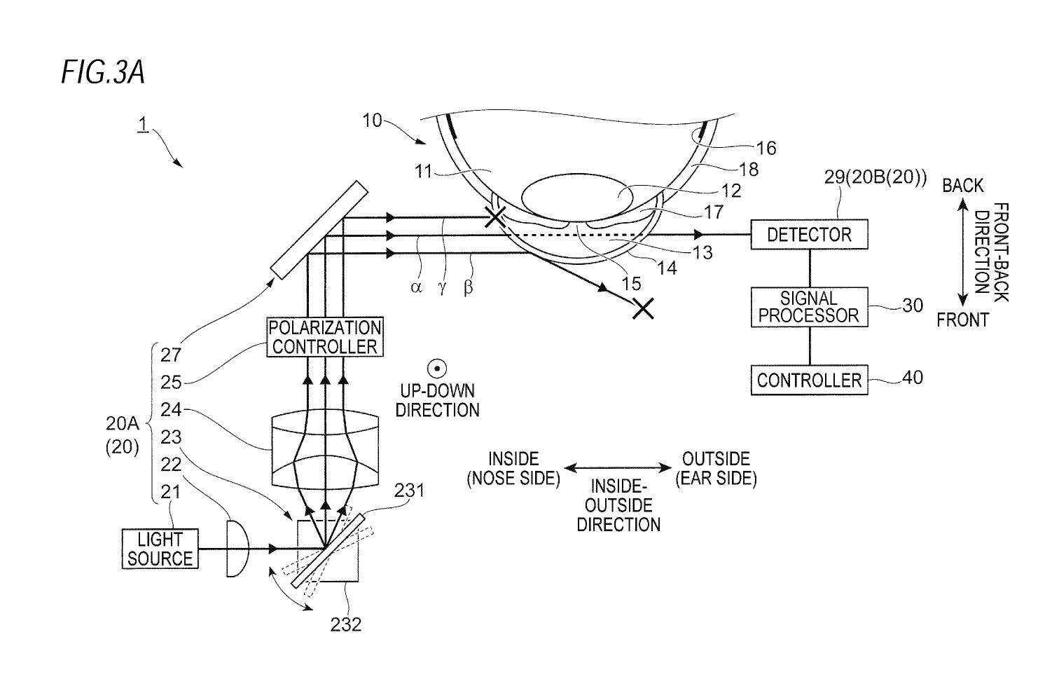

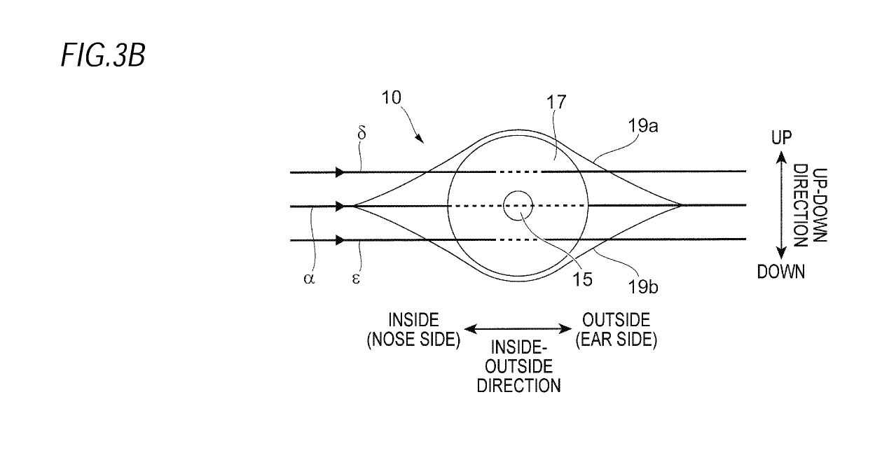

FIGS. 3A and 3B are views illustrating an exemplary configuration of the optical measurement apparatus 1 for an eyeball to which the third exemplary embodiment is applied. FIG. 3A is a view obtained when the eyeball 10 is viewed from the top side (a cross-sectional view in the upward and downward directions), and FIG. 3B is a view obtained when the eyeball 10 is viewed from the front side. The parts similar to those of the optical measurement apparatus 1 to which the second exemplary embodiment is applied (the optical measurement apparatus 1 to which the first exemplary embodiment is applied, except for some parts) are designated by the same reference numerals, and descriptions thereof will be omitted.

The optical measurement apparatus 1 to which the third exemplary embodiment is applied has a polarization controller 25 in addition to the parts of the optical measurement apparatus 1 to which the second exemplary embodiment is applied. The polarization controller 25 is an example of a polarization control part.

The polarization controller 25 includes a polarizer and a waveplate. Further, the polarization controller 25 extracts a predetermined polarized light (linearly polarized light, elliptically polarized light, circularly polarized light, etc.) from light projected from the light source 21.

When the light is reflected by the mirror 27, the reflectivity of a component (P) parallel to an incident surface and the reflectivity of a component (S) perpendicular to the incident surface depend on the refractive index and the incident angle of the mirror 27, respectively. For this reason, when the polarized light enters the mirror 27, the polarization state of the reflected light is changed by the incident angle in some cases. For example, in a case in which the linearly polarized light enters the mirror 27, the reflected light may also be linearly polarized at a certain incident angle, and the reflected light may be elliptically polarized at another incident angle.

Therefore, an incident angle to the mirror 27 may be fixed.

Therefore, similar to the second exemplary embodiment, the optical measurement apparatus 1 to which the third exemplary embodiment is applied is configured such that it is not necessary to consider a change in polarization state caused by a change in incident angle to the mirror 27 by using the telecentric optical system 24 including the telecentric fθ lens.

Similarly, when the polarized light passes through the lens, a polarization state is changed. Therefore, the polarization controller 25 is provided at a subsequent stage of the telecentric fθ lens of the telecentric optical system 24, that is, between the telecentric fθ lens and the mirror 27.

Here, the deflector 23 and the telecentric optical system 24 are also an example of a switching unit.

The detector 29 includes an analyzer or the like to detect an angle of optical rotation, as described below.

In a case in which a refractive index of the mirror 27, a polarization state (a direction of a vibrating surface, linear polarization, and elliptical polarization) of incident light, and an incident angle is already known, the polarization state of the reflected light may be calculated. Therefore, concentration of the optically active substance may be measured by using optical rotation by providing the polarization controller 25 in the optical measurement apparatus 1 for an eyeball to which the first exemplary embodiment is applied.

Because the operation of switching an incident position of light to the mirror 27 is similar to those described in the first exemplary embodiment and the second exemplary embodiment except that the concentration of the optically active substance is measured by using the optical rotation, a description thereof will be omitted.

### (Calculation of Concentration of Optically Active Substance)

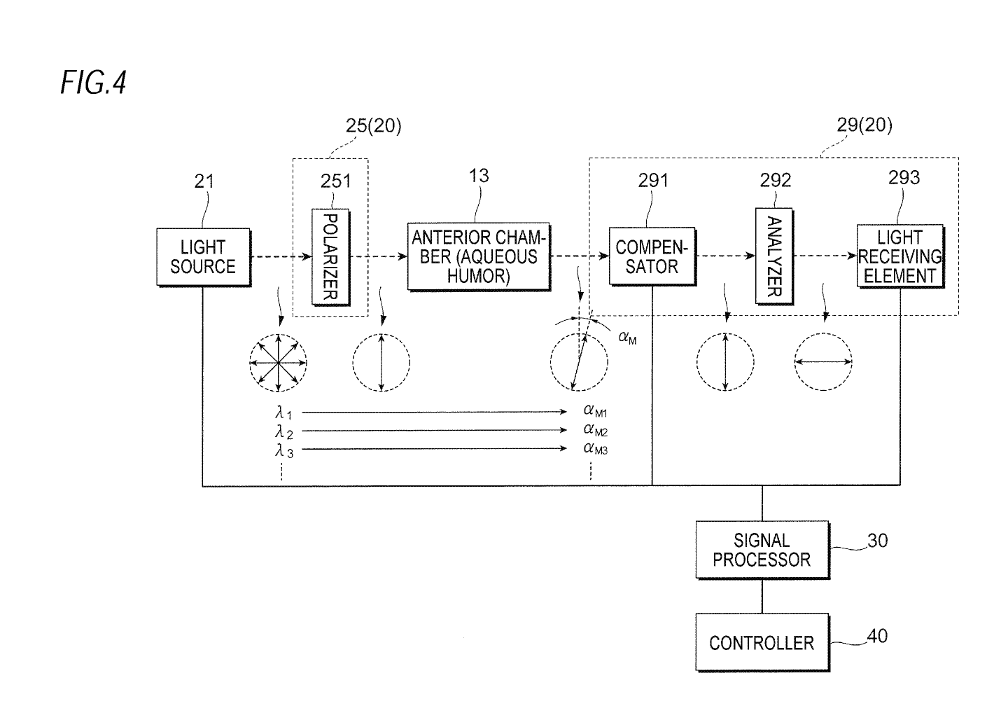

FIG. 4 is a view explaining a method of measuring, by the optical measurement apparatus 1, a rotation angle (optical rotation) of a vibrating surface caused by an optically active substance included in the aqueous humor in the anterior chamber 13. Here, for easy description, the optical path is configured not to be bent, and the telecentric optical system 24 and the mirror 27 are omitted.

It is assumed that the polarization controller 25 of the optical system 20 has a polarizer 251, and the detector 29 has a compensator 291, an analyzer 292, and a light receiving element 293.

Shapes of polarized lights, when viewed in a traveling direction of light, among the light source 21, the polarizer 251 of the polarization controller 25, the anterior chamber 13, and the compensator 291, the analyzer 292, and the light receiving element 293 of the detector 29, which are illustrated in FIG. 4, are indicated by arrows in circles, respectively.

The optical system 20 may have other elements (optical components, etc.).

The polarizer 251 is, for example, a Nicol prism or the like, and causes linearly polarized light from incident light, which has a predetermined vibrating surface, to pass therethrough.

The compensator 291 is, for example, a magneto-optical element such as a Faraday element using a garnet or the like, and rotates the vibrating surface of the linearly polarized light by a magnetic field.

The analyzer 292 is the same member as the polarizer 251, and allows the linearly polarized light having a predetermined vibrating surface to pass therethrough.

The light receiving element 293 is a silicon diode or the like, and outputs an output signal corresponding to intensity of light.

The light source 21 emits light having a random vibrating surface. Further, the polarizer 251 allows the linearly polarized light having a predetermined vibrating surface to pass therethrough. In FIG. 4, for example, the polarizer 251 allows linearly polarized light having a vibrating surface parallel to the drawing sheet to pass therethrough.

The vibrating surface of the linearly polarized light passing through the polarizer 251 is rotated by the optically active substance included in the aqueous humor in the anterior chamber 13. In FIG. 4, the vibrating surface is rotated by an angle α_{M} (optical rotation α_{M}).

Next, the vibrating surface, which is rotated by the optically active substance included in the aqueous humor in the anterior chamber 13, is returned back to the original state by the compensator 291. In a case in which the compensator 291 is the magneto-optical element such as the Faraday element, the vibrating surface of the light passing through the compensator 291 is rotated by applying a magnetic field to the compensator 291.

The linearly polarized light passing through the analyzer 292 is received by the light receiving element 293, and converted into an output signal corresponding to intensity of light.

Here, an example of a method of measuring the optical rotation α_{M} by the optical system 20 will be described.

First, in a state in which the light emitted from the light source 21 does not pass through the anterior chamber 13, the compensator 291 and the analyzer 292 are set by using the optical system 20 including the light source 21, the polarizer 251, the compensator 291, the analyzer 292, and the light receiving element 293 such that an output signal from the light receiving element 293 is minimized. In an example illustrated in FIG. 4, in a state in which light does not passes through the anterior chamber 13, the vibrating surface of the linearly polarized light passing through the polarizer 251 is orthogonal to the vibrating surface of the light passing through the analyzer 292.

Next, the light passes through the anterior chamber 13. Then, the vibrating surface is rotated by the optically active substance included in the aqueous humor in the anterior chamber 13. For this reason, an output signal from the light receiving element 293 exceeds the minimum value. Therefore, the vibrating surface is rotated by applying a magnetic field to the compensator 291 so that an output signal from the light receiving element 293 is minimized. That is, the vibrating surface of the light projected from the compensator 291 becomes orthogonal to the vibrating surface of the light passing through the analyzer 292.

An angle of the vibrating surface rotated by the compensator 291 corresponds to the optical rotation α_{M} generated by the optically active substance included in the aqueous humor. Here, a relationship between the magnitude of the magnetic field applied to the compensator 291 and the angle of the rotated vibrating surface is known in advance. Therefore, the optical rotation α_{M} may be found from the magnitude of the magnetic field applied to the compensator 291.

Specifically, lights having plural wavelengths λ (wavelengths λ₁, λ₂, λ₃, . . . ) are emitted to the aqueous humor in the anterior chamber 13 from the light source 21, and thus the optical rotations α_{M} (optical rotations α_{M1}, α_{M2}, α_{M3}, . . . ) are obtained for each wavelength. The sets of the wavelengths λ and the optical rotations α_{M} are received by the signal processor 30, and thus the concentration of the optically active substance, which is desired to be obtained, is calculated.

Plural optically active substances are included in the aqueous humor, as described above. Therefore, the measured optical rotation α_{M} is a sum of the optical rotations α_{M} provided by the plural optically active substances. Therefore, it is necessary to calculate the concentration of the optically active substance (here, glucose), which is desired to be obtained, from the measured optical rotation α_{M}. Because a publicly known method may be used to calculate the concentration of the optically active substance which is desired to be obtained, a description thereof will be omitted.

In FIG. 4, the vibrating surface of the polarizer 251 is parallel to the page surface, and the vibrating surface before passing through the analyzer 292 is perpendicular to the drawing sheet. However, in a case in which the vibrating surface is rotated by the compensator 291 in a state in which the light emitted from the light source 21 does not pass through the anterior chamber 13, the vibrating surface before passing through the analyzer 292 may be inclined with respect to a surface parallel to the drawing sheet. That is, the compensator 291 and the analyzer 292 may be set such that an output signal from the light receiving element 293 is minimized in a state in which light does not pass through the aqueous humor in the anterior chamber 13.

An example in which the compensator 291 is used as a method of obtaining the optical rotation α_{M} is described herein, but the optical rotation α_{M} may be obtained by using a component other than the compensator 291. Further, an orthogonal polarizer method (provided that the compensator 291 is used), which is the most basic measuring method of measuring the rotation angle (optical rotation α_{M}) of the vibrating surface, is described herein, but other measuring methods such as a rotating analyzer method, a Faraday modulation method, and an optical delayed modulation method may be applied.

### Fourth Exemplary Embodiment

In the optical measurement apparatus 1 to which the third exemplary embodiment is applied, the angle of light entering the mirror 27 is fixed by using the telecentric fθ lens for the telecentric optical system 24. In the optical measurement apparatus 1 to which the fourth exemplary embodiment is applied, the optical path is switched by moving the mirror 231 of the deflector 23 instead of using the telecentric optical system 24.

In the fourth exemplary embodiment, the concentration of the optically active substance such as glucose is measured by providing the polarization controller 25 and using an optical rotation. Further, the concentration of the optically active substance such as glucose may be measured by concentration without using the polarization controller 25.

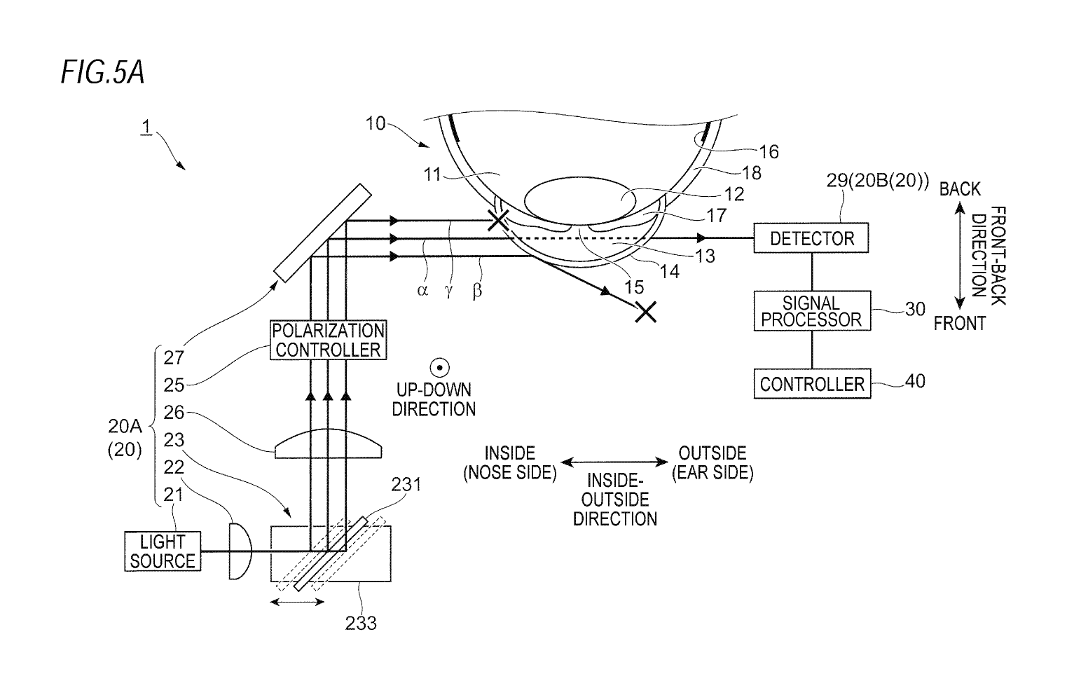

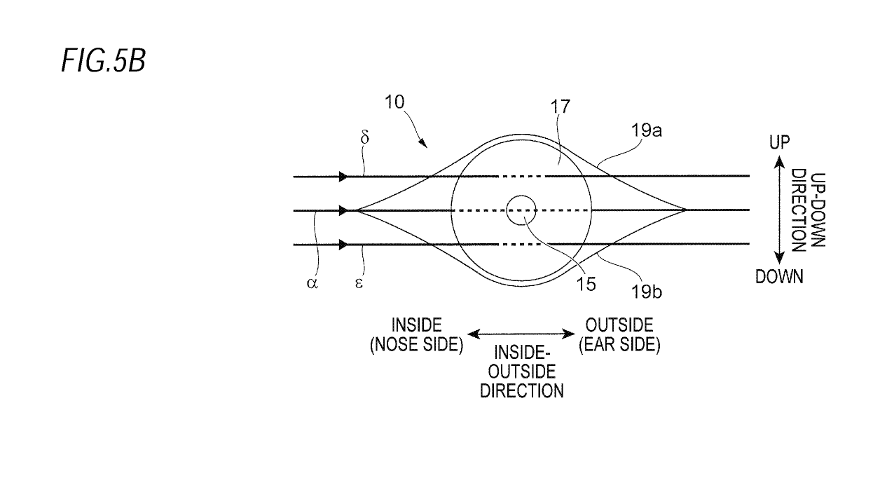

FIGS. 5A and 5B are views illustrating an example of a configuration of the optical measurement apparatus 1 for an eyeball to which the fourth exemplary embodiment is applied. FIG. 5A is a view illustrating the eyeball 10 when viewed from the top side (a cross-sectional view in the upward and downward directions), and FIG. 5B is a view illustrating the eyeball 10 when viewed from the front side. The parts similar to those of the optical measurement apparatus 1 to which the third exemplary embodiment is applied (the optical measurement apparatus 1 to which the first exemplary embodiment is applied, except for some parts) are designated by the same reference numerals, and descriptions thereof will be omitted.

The optical measurement apparatus 1 to which the fourth exemplary embodiment is applied is provided with a condensing lens 26 instead of the telecentric optical system 24. Further, the deflector 23 includes a mirror 231 and a linear motion stage 233 on which the mirror 231 is mounted so that the mirror 231 is moved in one direction by the linear motion stage 233. The linear motion stage 233 is an example of a moving unit.

That is, the reflecting surface of the mirror 231 is moved by the linear motion stage 233 in a direction of the optical path (the front-back direction in which light travels) Therefore, an incident position of light to the mirror 27 is switched. Further, an optical path is set to the state of the optical path α suitable to measure the concentration of the optically active substance included in the aqueous humor in the anterior chamber 13. That is, the optical path is set to pass across the anterior chamber 13.

Here, the deflector 23 and the condensing lens 26 are also an example of a switching unit.

In the fourth exemplary embodiment, an incident position of light to the mirror 27 is restricted by a movement direction of the linear motion stage 233. That is, an incident position of light to the mirror 27 is switched in a one-dimensional direction. For example, in FIG. 5A, the optical path is restricted by a movement of the face in the front-back direction.

Therefore, in a case in which the optical path is moved in the up-down direction of the face as illustrated in FIG. 5B, the light source 21 and the collimator lens 22 are disposed in a direction perpendicular to the drawing sheet, the movement direction of the linear motion stage 233 is also set to a direction perpendicular to the drawing sheet, and a direction of the mirror 231 on the linear motion stage 233 is set such that the light emitted from the light source 21 through the collimator lens 22 is reflected to the mirror 27, as illustrated in FIG. 5A.

A piezo element may be attached to the back surface of the mirror 231, and the front surface of the mirror 231 may be moved instead of using the linear motion stage 233. In this case, the linear motion stage 233 may be a driving device for operating the piezo element.

### Fifth Exemplary Embodiment

In the optical measurement apparatus 1 for an eyeball to which the fifth exemplary embodiment is applied, a circumference of the anterior chamber 13 of the eyeball 10 is immersed in a liquid. This state is called liquid immersion in some cases.

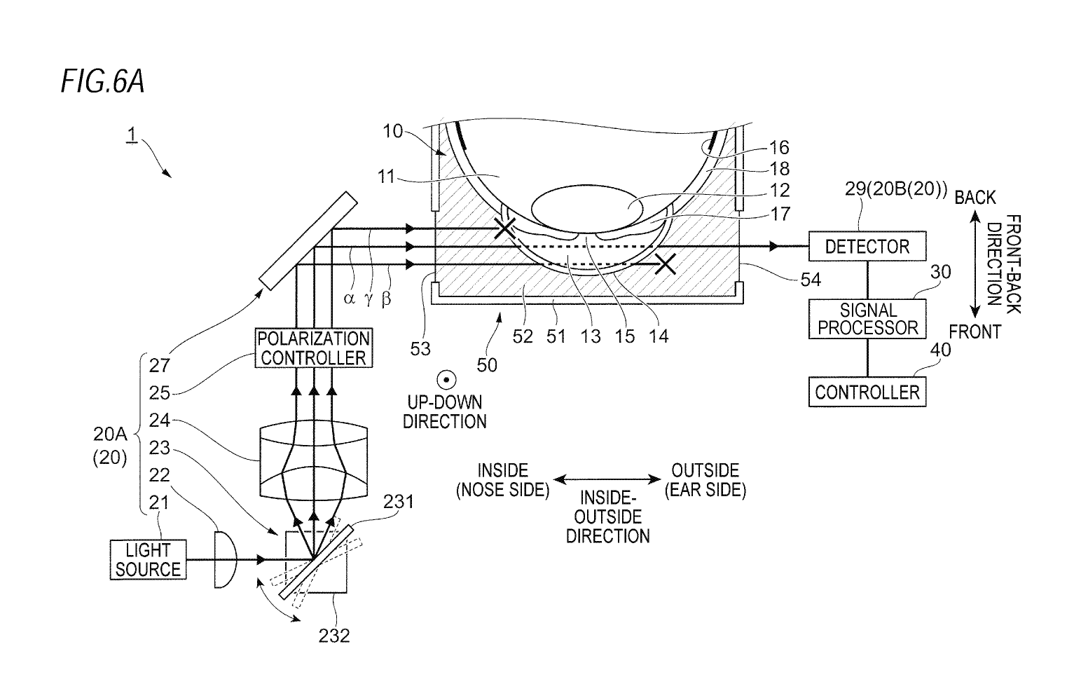

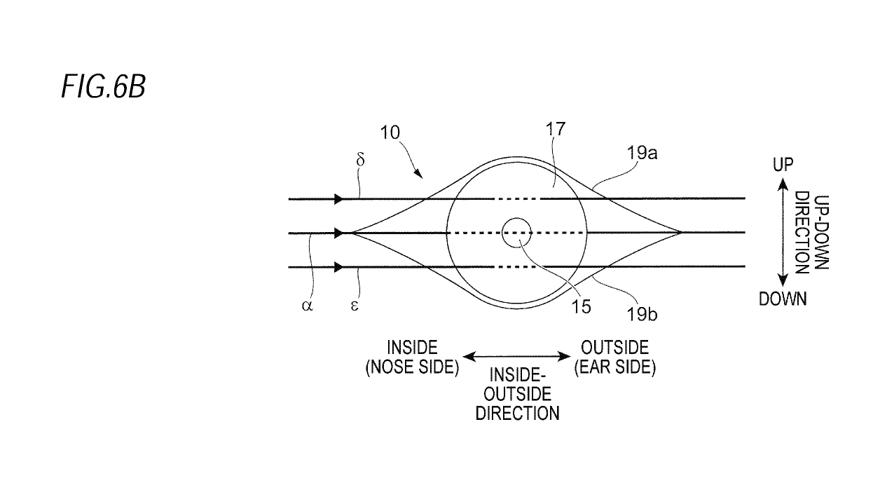

FIGS. 6A and 6B are views illustrating an exemplary configuration of the optical measurement apparatus 1 for an eyeball to which the fifth exemplary embodiment is applied. FIG. 6A is a view obtained when the eyeball 10 is viewed from the top side (a cross-sectional view in the upward and downward directions), and FIG. 6B is a view obtained when the eyeball 10 is viewed from the front side. Further, the configuration of the optical measurement apparatus 1, except for a liquid immersion part 50 to be described below, is identical to that in the third exemplary embodiment illustrated in FIGS. 3A and 3B. Therefore, the same parts are designated by the same reference numerals, descriptions thereof will be omitted, and different parts will be described.

The liquid immersion part 50 includes a container 51, and a liquid 52 that fills the container 51. The container 51 of the liquid immersion part 50 is moved close to a surface of the face at the periphery of the eyeball 10, so that the circumference of the anterior chamber 13 of the eyeball 10 is immersed in the liquid 52. The liquid 52 may have a refractive index that is less different from the refractive index of the aqueous humor. For example, water, saline solution, or the like may be used.

The liquid immersion part 50 includes an incident window 53 and an emission window 54, through which light passes, at portions corresponding to the optical path of the container 51 so that the light passes through the anterior chamber 13 while traversing the anterior chamber 13. The incident window 53 is configured such that the light reflected by the mirror 27 enters in a direction perpendicular to the incident window 53, and the emission window 54 is configured such that the light passing through the liquid 52 and the anterior chamber 13 is projected in a direction perpendicular to the emission window 54. Further, a size or a shape of the container 51 is not particularly limited as long as an incident position of light at the circumference (e.g., the cornea 14) of the anterior chamber 13 of the eyeball 10 is immersed in the liquid 52.

As described above, the liquid immersion part 50 inhibits the light reflected by the mirror 27 from being refracted by the surface of the cornea 14, thereby inhibiting the direction of the light from being changed. That is, a shape of the cornea 14 or the like hardly affects the light, so that the optical path traversing the anterior chamber 13 is easily set. Further, the optical path β runs without being reflected by the surface of the cornea 14, but the distance of the optical path β, which passes through the anterior chamber 13, is short.

The liquid immersion part 50 may be applied to the optical measurement apparatus 1 for an eyeball to which the other exemplary embodiments are applied.

### Sixth Exemplary Embodiment

In the optical measurement apparatus 1 for an eyeball to which the second to fourth exemplary embodiments are applied, the mirror 27 is set to have a predetermined incident angle. Further, the mirror 27 is disposed to be spaced apart from the eyeball 10.

In the sixth exemplary embodiment, the mirror 27 is provided on a contact member 60 having a mirror used in a state of being in contact with the surface of the eyeball 10. The contact member 60 having the mirror is an example of a mounting member.

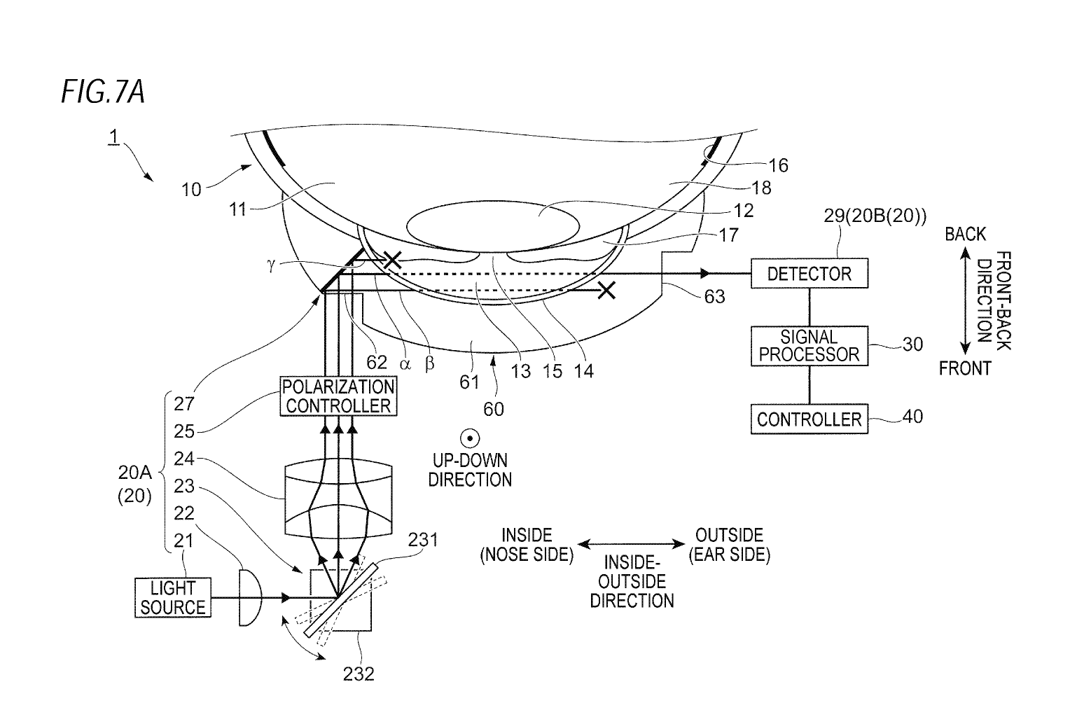

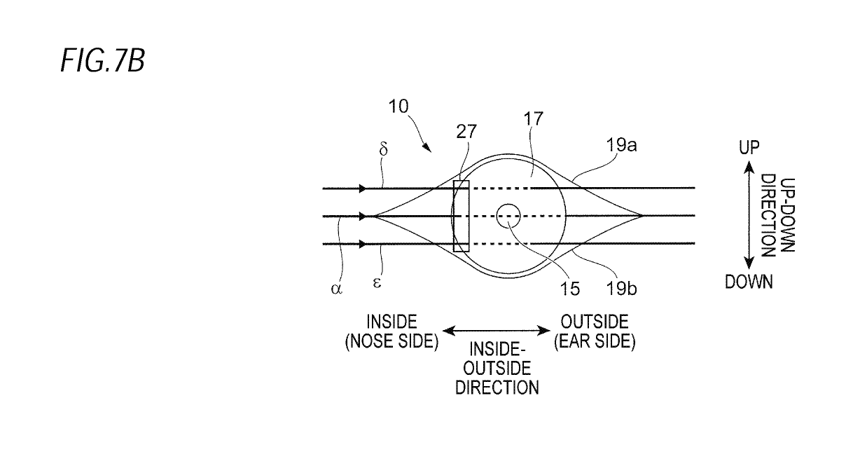

FIGS. 7A and 7B are views illustrating an exemplary configuration of the optical measurement apparatus 1 for an eyeball to which the sixth exemplary embodiment is applied. FIG. 7A is a view obtained when the eyeball 10 is viewed from the top side (a cross-sectional view in the upward and downward directions), and FIG. 7B is a view obtained when the eyeball 10 is viewed from the front side. Further, the configuration of the optical measurement apparatus 1, except for the contact member 60 having the mirror, which will be described below, is identical to that in the third exemplary embodiment illustrated in FIGS. 3A and 3B. Therefore, the same parts are designated by the same reference numerals, descriptions thereof will be omitted, and different parts will be described.

As illustrated in FIG. 7A, the contact member 60 having the mirror is a member for an eyeball such as so-called contact lens, and the contact member 60 is mounted on the surface (eyeball surface) of the cornea 14 of the eyeball 10. Further, the configuration in which the contact member 60 is mounted on the surface (eyeball surface) of the cornea 14 of the eyeball 10 is expressed herein as the configuration in which the contact member 60 is mounted on the eyeball 10.

The contact member 60 having the mirror has a mirror 27 that is provided in a base body 61.

The base body 61 is made of resin such as, for example, polyhydroxyethylmethacrylate, polymethylmethacrylate, silicone copolymers, or fluorine-containing compounds. In a case in which a refractive index of the base body 61 is close to refractive indexes of the aqueous humor in the anterior chamber 13 and the cornea 14 of the eyeball 10, refraction is inhibited at an interface between the contact member 60 having the mirror and the eyeball 10. Therefore, it is easy to set the optical path traversing the anterior chamber 13 of the eyeball 10. Further, the optical path β runs without being reflected by the surface of the cornea 14, but the distance of the optical path β, which passes through the anterior chamber 13, is short.

A portion of the base body 61, through which light enters toward the mirror 27, is configured as a flat surface 62 perpendicular to the light. In addition, a portion of the base body 61, through which light is projected toward the detector 29, is configured as a flat surface 63 perpendicular to the light. Therefore, when the light enters the contact member 60 having the mirror and the light is projected from the contact member 60 having the mirror, the optical path is inhibited from being bent due to the refraction of the base body 61.

As illustrated in FIG. 7B, the mirror 27 has a quadrangular external shape. Further, the external shape of the mirror 27 may be other shapes such as an arc shape.

The base body 61 needs not have a circular shape, and may have other shapes such as a quadrangular shape as long as the base body 61 may be mounted on the cornea 14.

The contact member 60 having the mirror, which is described in the sixth exemplary embodiment, may be applied to the second to fourth exemplary embodiments.

While various exemplary embodiments have been described above, these exemplary embodiments may be combined with each other.

The present disclosure is not limited to the aforementioned exemplary embodiments, but may be implemented in various forms without departing from the subject matter of the present disclosure.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An optical measurement apparatus for an eyeball, the optical measurement apparatus comprising:
a light reflecting unit that reflects light in a direction where the light passes across an anterior chamber of the eyeball; and
a switching unit that switches an incident position of the light to the light reflecting unit so as to inhibit the light from being moved from a state in which the light passes across the anterior chamber.

2. The optical measurement apparatus according to claim 1, wherein
the light reflecting unit is configured such that an incident angle of the light is set to a predetermined angle, and
the switching unit includes:
a reflective member that reflects the light,
an angle change unit that changes a reflection angle of the reflective member with respect to the light, and
a telecentric optical system that allows the light reflected by the reflective member to pass therethrough so that the light is projected to the light reflecting unit.

3. The optical measurement apparatus according to claim 2, further comprising:
a polarization controller that polarizes the light to a predetermined polarized light.

4. The optical measurement apparatus according to claim 3, wherein the polarization controller is disposed between the telecentric optical system of the switching unit and the light reflecting unit.

5. The optical measurement apparatus according to claim 2, wherein the reflective member of the switching unit changes the reflection angle with respect to the light in one direction of a reflecting surface and in a direction orthogonal to the one direction.

6. The optical measurement apparatus according to claim 1, wherein
the light reflecting unit is configured such that an incident angle of the light is set to a predetermined angle, and
the switching unit includes
a reflective member that reflects the light, and
a moving unit that moves the reflective member in a front-back direction in which the light travels.

7. The optical measurement apparatus according to claim 6, further comprising:
a polarization controller that polarizes the light to a predetermined polarized light.

8. The optical measurement apparatus according to claim 1, further comprising:
a container that allows a circumference of the anterior chamber of the eyeball to be immersed in a liquid.

9. The optical measurement apparatus according to claim 2, wherein the light reflecting unit is provided in a mounting member that is used in a state of being in contact with a surface of the eyeball.

10. An optical measurement apparatus for an eyeball, comprising:
a light reflecting unit that reflects light in a direction that passes across an anterior chamber of the eyeball; and
a switching unit that switches an incident position of the light to the light reflecting unit such that the light passing across the anterior chamber moves in parallel in at least one of a front-back direction and an up-down direction of the eyeball.
